(19) United States
(12) Patent Application Publication
(10) Pub. No.: US 2006/0025659 A1
(43) Pub. Date: Feb. 2, 2006

(54) Optical measuring device for substances in vivo

(76) Inventors: Masashi Kiguchi, Kawagoe (JP); Tatsuya Tomaru, Hatoyama (JP); Hideaki Koizumi, Tokyo (JP); Atsushi Maki, Fuchu (JP); Hideo Kawaguchi, Hatoyama (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/069,963

(22) Filed: Mar. 3, 2005

(30) Foreign Application Priority Data
Aug. 2, 2004 (JP) 2004-225166

(51) Int. Cl. A61B 5/00

(52) U.S. Cl. 600/316

(57) ABSTRACT

In order to provide a compact device easy to handle and adjust for use in bloodless measurement of the glucose concentration, in which the angle of polarization varies in synchronism with the magnetic field modulation, the direction of applying the magnetic field is so arranged as to cross the optical axis.

### CLAIM OF PRIORITY

The present application claims priority from Japanese application JP 2004-225166 filed on Aug. 2, 2004, the content of which is hereby incorporated by reference into this application.

### FIELD OF THE INVENTION

The present invention relates to noninvasive and bloodless measurement of the concentrations of substances, especially that of glucose, in vivo by using light rays, and more particularly to a glucose sensor and a glucose monitor.

### BACKGROUND OF THE INVENTION

Diabetes patients have to receive regular checkup of the glucose concentration in blood for blood sugar control. A blood sample would allow measurement of the glucose concentration by the enzyme electrode method or otherwise, but taking a blood sample causes pain to the patient. For this reason, development of a bloodless measuring device which requires no blood sampling is called for.

On the other hand, a glucose sensor using Faraday effect is disclosed in Patent Reference 1. This glucose sensor disclosed in Patent Reference 1 detects glucose by utilizing the phenomenon that application of a magnetic field to the blood or urine sample causes a linearly polarized light to rotate. The glucose sensor disclosed in Patent Reference 1 measures liquid, such as sampled blood or urine, contained in a cell, and therefore requires blood sampling for measurement. It allows no bloodless measurement.

Patent Reference 1: Pamphlet of International Publication No. WO00/60350

### SUMMARY OF THE INVENTION

An object of the present invention is to provide a device for bloodless measurement of the glucose concentration in blood by utilizing Faraday effect.

Hereupon, a brief description of Faraday effect, on which the invention is based, is described. When a magnetic field H is being applied to a medium, if a linearly polarized light passes the medium, the polarized light is rotated by an angle α. This phenomenon is known as Faraday effect, and this angle α, as Faraday rotation angle α. With the angle formed by the magnetic field H and the optical axis being represented by θ, the Verdet constant of the medium by V and the optical path length by L, the relationship represented by Equation (1) holds among the Faraday rotation angle α and these values.
α=VHL cos θ (1)

Therefore, the Verdet constant V of the medium can be figured out by observing the Faraday rotation angle α. Since the Verdet constant V is proportional to the concentration of any Faraday-active substance, the glucose concentration can be figured out in this way.

Since the Faraday rotation angle α is proportional to the cosine (cos θ) of the angle θ formed by the magnetic field H and the optical axis, usually an optical system or a magnet is so arranged as to make the optical axis and the magnetic field H parallel to each other to maximize α. For this reason, frequently a hole is bored into the magnet and the sample is arranged in the hole through which the light passes. This requires the sample to be put into a cell for measurement, which accordingly cannot be done bloodlessly, and moreover the freedom of arrangement of the optical system or the magnet is restricted, making it difficult to design its arrangement properly or to necessitate a large size for the device.

In order to solve this problem, a device is comprised of means to irradiate the living body to be checked with a light, means to detect the resultant transmitted or reflected light, means to apply a magnetic field to lights passing the living body in a direction crossing the position of irradiation with light and the position of detecting the light, and means to analyze the polarization of the detected light, whereby the Faraday rotation angle α is measured, from which the glucose concentration is bloodlessly determined. If a plurality of independent lights differing in wavelength are used in this process, the glucose concentration can be measured even more accurately.

### BRIEF DESCRIPTION OF THE DRAWINGS

FIG. 1 is a block diagram illustrating the principle of a transmission type concentration measuring device for substances in vivo using Faraday effect.

FIG. 2 is a block diagram illustrating the principle of a reflection type concentration measuring device for substances in vivo using Faraday effect.

FIG. 3 is a block diagram illustrating the principle of a concentration measuring device for substances in vivo using the effect of interference by a low-coherence light source and Faraday effect.

### DESCRIPTION OF THE PREFERRED EMBODIMENTS

The present invention concerns living bodies as the object of checkup, and more particularly to the observation of the scattering or reflection of lights injected into a living body through its surface. Since the optical axes of lights scattered in a living body may travel in a variety of directions irrespective of the direction in which the lights are brought to incidence into the body or the direction in which the lights emitted from the body are detected, there is no need to arrange the line connecting the point of incidence and the point of detection in parallel to the magnetic field H. Conversely, even if the line connecting the point of incidence and the point of detection is orthogonal to the magnetic field H, the angle θ formed by the magnetic field H and the optical axis in the living body will not be equivalently 90 degrees, and the Faraday rotation angle α will not be 0. In this case, cos θ will be space-averaged to ⅓.

The present invention is a proposal, taking note of this point, for a device in which the compact arrangement of the optical system and of the magnet is made easy to handle and adjust.

Faraday effect is not the only phenomenon for linearly polarized lights to rotate, but rotary polarization requiring no magnetic field application is another example, and glucose also has such optical activity, i.e., chirality. However, when lights travel in the inverse direction on the same path in a scatterer, Faraday effect will double the rotation angle α because the polarized lights are rotated in the same direction, but rotary polarization will reduce the rotation angle to 0 because the polarized lights rotated in the reverse direction. Therefore, it is more effective to perform measurement by using Faraday effect where it concerns a living body in which scattering is intense. This can also be said of observing Faraday effect by using optical coherence tomography (OCT) to be described afterwards, by which lights reflected in a living body are selectively observed.

Furthermore, there also are other substances in vivo than glucose that have Faraday effect including blood and water. For accurate measurement of the glucose concentration, some way or other has to be devised to cancel the contributions of these obstructing substances. In order to cancel the contributions of these obstructing substances in vivo having Faraday effect, i.e. blood, water and the like, the use of one or another of the following methods can be effective.

Lights of multiple wavelengths are used for measurement, and the Faraday rotation angle and absorbed quantity of the light of each wavelength are measured. The product of the concentration and the optical path length L in the living body will be referred to as the concentration length in the following description. The concentrations of oxygenated hemoglobin and deoxygenated hemoglobin, the concentration length of water, and the scattering term can be figured out from the absorbed quantities of lights of at least four wavelengths by solving simultaneous equations regarding these waveforms by using modified Lambert-Beer's equations (Equation (2) and Equation (3)).

$$\begin{matrix}ln \frac{I ( λ ) [ L ]}{I ( λ ) [ 0 ]} = \{ {ɛ}_{g} ( λ ) {C}_{g} + {ɛ}_{o} ( λ ) {C}_{o} + {ɛ}_{d} ( λ ) {C}_{d} + {ɛ}_{h} ( λ ) {C}_{h} \} L + G & ( 2 ) \\ α ( λ ) = \{ {V}_{g} ( λ ) \cdot {C}_{g} + {V}_{o} ( λ ) \cdot {C}_{o} + {V}_{d} ( λ ) \cdot {C}_{d} + {V}_{h} ( λ ) \cdot {C}_{h} \} \cdot \frac{H L}{3} & ( 3 )\end{matrix}$$

In these equations, I(λ)[L] represents the intensity of the light of a wavelength λ when it has traveled by a path length L; εg(λ), εo(λ), εd(λ) and εh(λ), the molecular absorption constants of glucose, oxygenated hemoglobin, deoxygenated hemoglobin and water at the wavelength λ; Cg, the glucose concentration; Co, the oxygenated hemoglobin concentration; Cd, the deoxygenated hemoglobin concentration; and Ch, the concentration of water. Vg(λ), Vo(λ), Vd(λ) and Vh(λ) are the respective Verdet constants of glucose, oxygenated hemoglobin and deoxygenated hemoglobin per unit concentration of water at the wavelength λ. G represents the scattering term, i.e. the luminous energy dissipated by scattering or otherwise.

By measuring the rotation angle α(λ) at four wavelengths for instance, the product of the glucose concentration Cg and the optical path length L in the living body can be figured out. Estimation of L by comparison with the result of measurement done in advance by some other method, such as the enzyme electrode method, would give the glucose concentration Cg. Ideally, this calibration of the optical path length L in the living body should be performed for each patient, but it is acceptable for practical purposes to have it represented by the average optical path length L based on figures obtained from many patients for each region of measurement.

Another method of figuring out the glucose concentration Cg will be described hereupon. The glucose concentration Cg can be stated in the form of Equation (4) as the function of the rotation angle α(λn) of the wavelength λn and the absorbed
C_{g}=ƒ{α(λ1), A(λ1), α(λ2), A(λ2), α(λ3), A(λ3), - - - , α(λn), A(λn)} (4)
quantity A (λn) of the light of that wavelength.

This equation will now be expanded into a series by using an expansion coefficient. Since the expansion coefficient can be found out in advance by fitting based on comparison with the result of measurement done by some other method, such as the enzyme electrode method, it is possible to obtain the glucose concentration Cg from the measured value α(λn) and A (λn).

Where a method based on Equation (2), Equation (3) or Equation (4) is used, the result is affected by substances contained in other tissues than blood vessels, such as skins. If signals can be separated in the depthwise direction, glucose in blood can be measured efficiently. Measurement with signals separated in the depthwise direction can be accomplished by the space separation method or optical coherence tomography (OCT).

The space separation method is a technique by which separation in the depthwise direction is performed on the basis of results obtained by differentiating the distance between the irradiated position on the object region and the position of detection. This method derives from the knowledge that the sensitivity of detection in deep parts increases with the expansion in distance between the irradiated position and the detection position. Details of the space separation method are described in Proceedings of SPIE, vol. 3597, p. 582-592, 1999.

OCT is a method by which a low-coherence light source is used, the reflected light from object region and a reference light are caused to interfere with each other with a Michelson interferometer or the like, and the interfering components alone are detected. Since scattered light is not significantly interferential, its influence can be eliminated. In this process, sweeping in the depthwise direction of the object region is equivalently accomplished usually by sweeping the optical path length on the reference light side. Details of OCT are described, for instance, in the Japanese Patent Applications Laid-Open Nos. 2003-144421 and 2003-543 and references cited therein.

By using the OCT technique, glucose concentration in deep parts can be selectively obtained.

While the optical path length is extended by scattering in the living body and the signal intensity is increased where the space separation method is used, the variations in polarization accompanying scattering pose an obstructive factor. In this respect, the OCT technique has an advantage of making possible measurement free from the influence of scattering.

By observing the rotation of polarized light by using these techniques, the Verdet constant and the glucose concentration can be figured out.

(Embodiment 1)

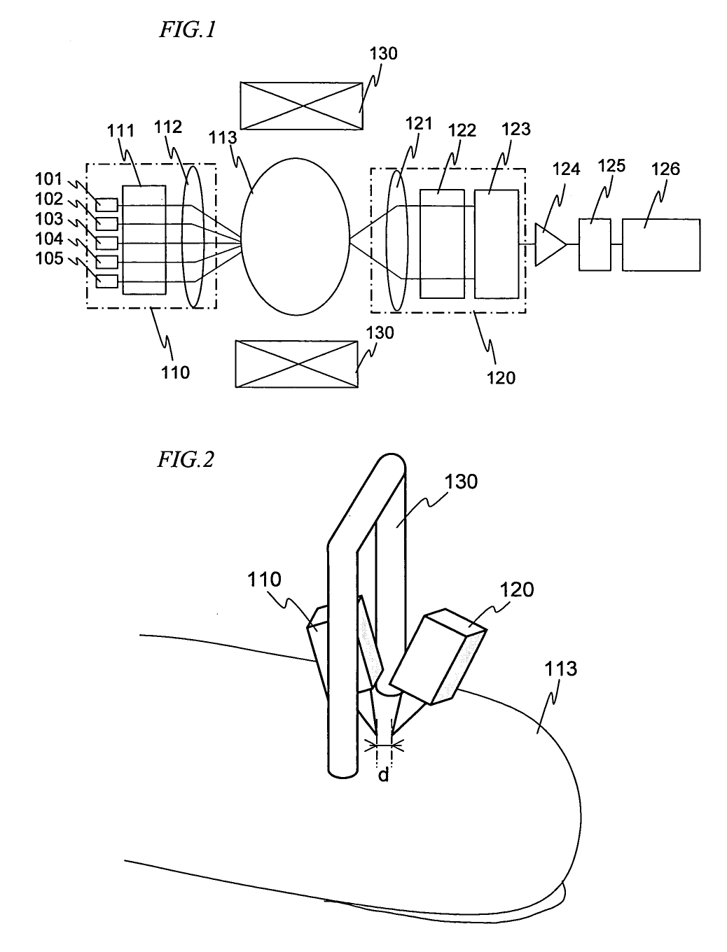

A first preferred embodiment of the present invention will be described with reference to FIG. 1. FIG. 1 is a block diagram illustrating the principle of a transmission type concentration measuring device for substances in vivo using Faraday effect. Reference numerals 101 through 105 denote laser diodes of 780 nm, 830 nm, 870 nm, 1300 nm and 1500 nm, respectively, in wavelength.

The laser diodes are amplitude-modulated with different frequencies fi, respectively 10 kHz, 12 kHz, 14 kHz, 16 kHz and 18 kHz, for example. Reference numeral 111 denotes a lens and 112, a polarizer. The laser diodes 101 through 105, the lens 111 and the polarizer 112 constitute a light source system 110.

The output lights of the laser diodes 101 through 105 are collimated by using the lens 111 and, after passing the polarizer 112, irradiate an object region (e.g. a finger) 113. The lights having passed the object region 113, after being collimated by a lens 121, are guided to an analyzer 122. Though the laser diodes 101 through 105 are illustrated as constituting a single row in FIG. 1 for the convenience of understanding, in practice it is more preferable to arrange them two-dimensionally on a plane substantially parallel to the lens surface in the vicinity of the center line of the lens 111. The analyzer 122 is off a right angle to the polarizer 112 by 5 degrees. The lights having passed the analyzer 122 undergo photoelectric conversion by a photodiode 123. The lens 121, the analyzer 122 and the photodiode 123 constitute a detector system 120.

The output current of the photodiode 123, after undergoing current/voltage conversion by a preamplifier 124, is input to an AD converter 125. An AC magnetic field of frequency F is already applied to the object region 113 with an electromagnet 130. F is supposed to be 200 Hz here. Therefore, the variation of the angle of polarization in synchronism with magnetic field modulation can be observed as the luminous energy variation for each wavelength fi by having a data processor 126 perform synchronous detection of the output signals of the AD converter 125 with the amplitude modulation frequency fi of the laser diodes+the magnetic field modulation frequency F as the reference signal. By observing the variations of the angle of polarization synchronized with the magnetic field modulation, the angle of polarization due to Faraday effect can be extracted in isolation from variations of polarization due to scattering and Chirality, etc., and the glucose concentration can be calculated by using the technique described above.

Although laser diodes are used as light sources in this embodiment, light emitting diodes can as well be used instead. Also, while wavelengths are separated by synchronous detection by subjecting the light sources to amplitude modulation, they can be separated on a time axis by driving the diodes with pulses to achieve light emission sequentially. The extraction of synchronous components is facilitated in this case by making the frequency F of the AC magnetic field applied by the electromagnet 130 sufficiently greater or sufficiently smaller than the aforementioned pulse frequency. Although lights from the sources are let propagate in the air to irradiate the object region as separated by wavelength, the output lights of the laser diodes can be mixed with fibers and let irradiate the object region. Where fibers are to be used, the polarizer 112 can be dispensed with by using polarization plane conserving fibers.

Although the object region can be inserted between the light source system 110 and the detector system 120, an electromagnetic coil can be arranged in their vicinity in this Embodiment 1, and a finger is cited as the example of region, the object region can as well be an earlobe, lip, cheek or the like.

(Embodiment 2)

A second preferred embodiment of the invention will now be described with reference to FIG. 2. FIG. 2 is a block diagram illustrating the principle of a reflection type concentration measuring device for substances in vivo using Faraday effect. While the arrangement of Embodiment 1 is to let lights pass the object region, this second embodiment uses a reflection type arrangement. The hardware configuration is almost the same as that of Embodiment 1. The light source system 110 and the detector system 120 are arranged on the same side with respect to the object region 113. Incidentally, the light source system 110 and the detector system 120 are the same as their respective counterparts in FIG. 1. The focal position of the lens 111 and that of the lens 121 may be the same, but deviating one from the other would increase the contribution of blood vessels within the epidermis to signals. For this reason, the distance between the focal positions of the two lenses here is set to 5 mm. The poles of the electromagnet 130 are so arranged as to have the magnet stride over the line connecting the focal position of the lens 111 and that of the lens 121.

Though not shown, if a holder is provided to hold the light source system 110, the detector system 120 and the electromagnet 130 in an integrated way, the user of the device can accomplish measurement by softly pressing it against any position of the object of checkup.

As Embodiment 2 uses a reflection type arrangement, the limitation imposed on the object region by light transmissivity is significantly reduced. Furthermore, as its user has only to grab the holder in his or her hand and press the measuring device against the object region, the limitation imposed by the object region is also eased. It provides the benefit of facilitating measurement on not only a finger but also a region of high blood vessel density, such as the inside of a cheek.

Although the description of Embodiments 1 and 2 supposed measuring at only one point, multi-point measurement or image measurement by using a plurality each of light source systems and detecting systems, spatial sweeping with a mirror, or using a two-dimensional photodiode or a television camera in place of the photodiode 123 would enable the blood vessel region to be compared with other regions, resulting in enhanced accuracy of measurement.

(Embodiment 3)

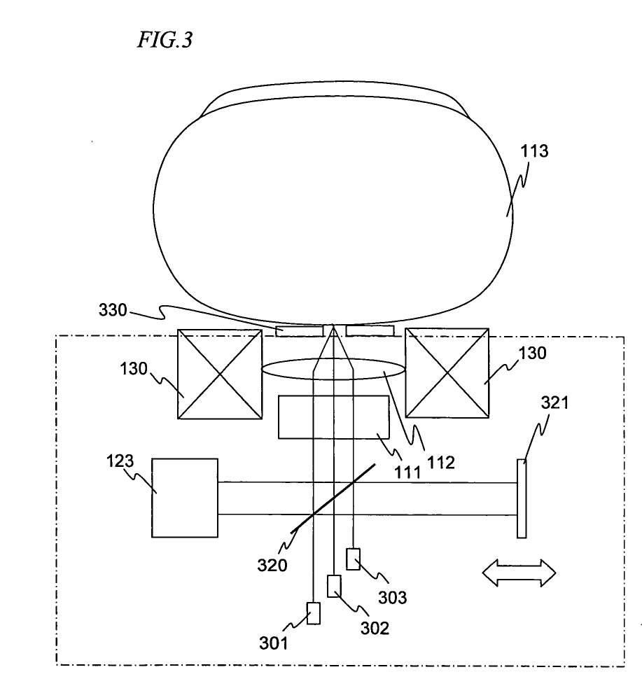

A third preferred embodiment of the invention will be described with reference to FIG. 3. FIG. 3 is a block diagram illustrating the interfering effect of low-coherence light sources and the principle of a concentration measuring device for substances in vivo using the effect of interference by low-coherence light sources and Faraday effect. The output lights of super-luminescent diodes 301, 302 and 303 of 840 nm, 1310 nm and 1550 nm in wavelength, after being let pass a half mirror 320 and a polarizer 111, are focuses on the surface of the object, for instance the surface of a finger 113, by using a lens 112. The reflected lights from the surface of the finger 113 are again collimated by the lens 112 and, after passing the polarizer 111, are reflected by the half mirror 320 to come into incidence on the photodiode 123. On the other hand, part of the lights emitted from the super-luminescent diodes 301 through 303 is separated by the half mirror 320, reflected by a mirror 321, and comes into incidence on the photodiode 123 through the half mirror 320. This is called the reference light. When the mirror 321 is swept in the directions of the arrow in the drawing, interference occurs when the optical path length of the reflected light from the surface of the finger 113 (the length of the optical path from the position on the surface of the finger 113 to the position of incidence on the photodiode 123 after reflection by the half mirror 320) and the reference light of the optical path length (the length of the optical path from the position where the light reflected by the half mirror 320, which is further reflected by the mirror 321, to the position of incidence on the photodiode 123) have become identical in the coherence range of each super-luminescent diode, resulting in a variation in the output signal of the photodiode 123. The optical path length of the reference light can be swept, for example, by moving the mirror 321 or arranging a prism in place of the mirror 321 and rotating it.

If it is supposed here that the distance between the super-luminescent diodes 301 through 303 and the surface of the finger 113 according to the wavelength, the optical path length in which the reflecting light from the same depth within the finger interferes will vary. In other words, the position in the mirror 321 where an interference signal is observed will differ. By setting this difference in the distance between the super-luminescent diodes 301 through 303 and the finger 113 to the twofold or more of the depth observable with a super-luminescent diode of one wavelength, the output signals from the photodiode 123 can be divided by wavelength. Whereas a magnet 130 applies a magnetic field mainly in the transverse direction of the drawing, Faraday effect will arise because of its optical axis component in the transverse direction if the focal distance of the lens 112 is shortened and intense focusing is done. A spacer 330 functions as an element to keep the distance between the lens 112 and the finger 113 substantially constant when the finger is pressed. The sweeping speed of the mirror 321 is set sufficiently slower or faster than the modulation speed of the magnetic field. For instance the magnetic field modulation frequency by the magnet 130 is set to 500 Hz and the sweeping frequency by the mirror 321, to 2 Hz.

Now will be described in specific terms the method by which the glucose concentration is figured out by this Embodiment 3. Although a large part of lights coming incident into a living body is intensely scattered, some part of them travels straight ahead, is reflected by surfaces having level gaps in refraction index, such as boundary faces of different layers in the living body, and returns straight suffering almost no scattering. Since such a light is not disturbed in phase, it is caused to interfere with the reference light by an optical interfering element provided outside. The magnitude I of this interfering component is expressed in Equation (5), where r is the reflection factor on the reflecting surface; β, the rate of attenuation inflicted by absorption and scattering as the light travels within the living body; α, the Faraday rotation angle; and Ii, the intensity
I=Iiβr cos α (5)
of light incident on the living body.

By applying the magnetic field H in a rectangular waveform by using the magnet 130, a state in which the magnetic field H is applied and a state in which it is not applied can be created alternately. The ratio between them can be represented by Equation (6), where I(H) is the intensity of the interfering component when the magnetic field H is applied and I(0), the
I(H)/I(0)=cos[VHL] (6)
intensity of the same when the magnetic field H is not applied.

As the intensity of the magnetic field H is known here and the optical path length L in the living body can be figured out as the product of the swept optical path length on the reference side and the refractive index of the living body (about 1.4), the Verdet constant V can be determined. Separation of the glucose from other components in vivo can be accomplished by the above-described method using Equation (4).

As OCT can give information in the depthwise direction, it is possible to observe variations in only the blood vessels underneath the epidermis. Thus the rotation angle of polarization can be measured of each of the lights reflected by the upper wall and the lower wall of the blood vessel, and accordingly the components attributable to blood can be extracted by figuring out the difference between them. This makes it possible to limit the contributions of water.

While the magnetic field H is supposed to have a rectangular waveform here, if it is a triangular wave, Equation (6) will give a sine wave, and therefore the sensitivity can be increased by lock-in detection.

Although output lights from the super-luminescent diodes are propagated in the air as illustrated in FIG. 3, optical fibers can as well be used instead in this example, where polarization plane conserving fibers can be used to serve as polarizers as well. Where fibers are to be used, if the optical path length is to be altered, in either case the fiber length can be varied according to the wavelength or a method of applying pressure to the fibers can also be used.

In Embodiment 3, since wavelength components are separated according to differences in optical path length, the light sources need no modulation, resulting in the benefit of simplifying the hardware.

(Other Embodiments)

While the embodiments described above are intended for measuring glucose, the concentration of hemoglobin, myoglobin or the like can be measured in accordance with the same principle and with the same hardware, which can also be used for measuring muscular activity and cerebral functions.

The invention contributes to increasing the freedom in designing the arrangement of the optical system and the magnet, and thereby makes possible realization of a compact device easy to handle and adjust, which would provide the benefit of relieving diabetes patients from the pain of daily blood sampling.

The invention, as it enables the glucose concentration in blood to be measured bloodlessly, can provide a glucose monitor which can be used both in clinical establishments and at home. It can also be developed into a wearable regular measuring device, which would alleviate the burden of measurement on patients.

Reference signs stated in the drawings have the following meanings, respectively.

101 through 105: Laser diodes, 111: polarizer, 112: lens, 113: finger, 121: lens, 122: analyzer; 123: photodiode, 124: preamplifier, 125: AD converter, 126: data analyzer, 301 through 303: low-coherence light sources, 320: half mirror, 321: mirror

What is claimed is:

1. An in vivo measuring device comprising:
means of irradiating with lights a position to be irradiated on the surface of a living body;
means of detecting said irradiating lights having passed the living body in a detection position on the surface of the living body;
means of applying a magnetic field to lights passing said living body in a direction crossing the line connecting said light-irradiated position and said light detection position;
means of analyzing the polarization of said detected lights; and
means of figuring out the concentrations of substances in vivo on the basis of said information on polarization.

2. The in vivo measuring device, as set forth in claim 1, wherein said light-irradiated position, said light detection position and said magnetic field applying means are on the same plane of the living body.

3. The in vivo measuring device, as set forth in claim 1 or 2, wherein the lights irradiating the surface of said living body are independent lights differing in wavelength, undergo amplitude modulation with different frequencies, the magnetic field applied to the lights passing said living body undergo frequency modulation, and said detected lights are subjected to synchronous detection with the sum of the amplitude modulation frequency of the lights and the modulation frequency of the magnetic field to observe variations in the angle polarization synchronized with the magnetic field modulation.

4. An in vivo measuring device comprising a plurality of low-coherence light sources having different wavelengths, means of branching lights from said low-coherence light sources, irradiating the surface of a living body with one branched component and guiding the other to a mirror to cause the reflected lights to interfere with each other to sweep the position of said mirror, and means of applying a magnetic field to the surface of the living body irradiated with said lights, wherein the distance between said low-coherence light sources and the living body is varied according to the wavelengths of said low-coherence light sources, the intensity of said magnetic field is varied to create a state in which the magnetic field is applied and a state in which the magnetic field is not applied, and variations in polarization are measured according to the intensity of the interfering component in each state.
